(19) United States
(12) Patent Application Publication
(10) Pub. No.: US 2026/0335491 A1
(43) Pub. Date: Oct. 29, 2026

(54) ESSENTIAL OIL FRAGRANCE DIFFUSER

(71) Applicant: Carly(HK) Limited, Foshan (CN)

(72) Inventor: Jierong XU, Foshan (CN)

(73) Assignee: Carly(HK) Limited, Foshan (CN)

(21) Appl. No.: 19/192,333

(22) Filed: Apr. 29, 2025

(51) Int. Cl. A61L 9/14

(52) U.S. Cl. CPC A61L 9/14; A61L 2209/133; A61L 2209/134

(57) ABSTRACT

The present invention discloses an essential oil fragrance diffuser. The present invention has the advantages of preventing liquid from flowing out and reducing noise through the matching arrangement of the spray head main body pipe, a first silencing member, a spray head cover, a second silencing member, a spray head body, a spray core, and a ball. According to an anti-backflow protection structure, when the product topples over, a steel ball rolls to a mist outlet hole by utilizing a principle of gravity, the mist outlet hole is blocked, so that the liquid can be prevented from flowing out from the mist outlet hole.

### TECHNICAL FIELD

The present invention relates to the technical field of aromatherapy machines, and in particular, to an essential oil fragrance diffuser.

### BACKGROUND

The essential oil fragrance diffuser is a device for diffusing essential oil molecules into the air by a physical method. The essential oil fragrance diffuser utilizes a two-fluid spray principle to convert essential oil into tiny particles through high-pressure spray of airflow air, and these tiny particles with high-concentration form negative ions with high-concentration in the air for emission, thereby achieving the effect of aromatherapy.

The fragrance diffuser generates negative pressure by using an air pressure difference, when the gas fluid flows through the micropore area in the pipeline, the flow velocity of the gas fluid is increased, the static pressure is reduced, negative pressure is generated on the air inside the bottle, the essential oil in the bottle is drained to a position below the high-pressure nozzle through the suction pipe, and the air column of the high-pressure nozzle is directly sprayed to the liquid, and the liquid is dispersed to form a mist shape; and only one circulating air channel is in communication with the outside in the closed spray head, and the scattered mist-shape liquid is driven by the high-pressure airflow to be blown out of the spray head along the air channel.

In the prior art, due to the particularity of the internal structure of the spray head, the distance of the airflow channel is shortened, the scattered mist-shape liquid is dispersed, and the mist blown out of the spray head is particularly sparse. When the high-pressure airflow is sprayed to the liquid through the nozzle, a high-pressure spraying sound is generated, and meanwhile, after the device topples over, liquid in the device can flow out of the spray head along the air channel, and the liquid does not spray for a long time, causing blockage at the spraying structure, and since the gas fluid and the liquid are sprayed oppositely in the nozzle with a tiny size, the mounting precision is relatively high, resulting in a low yield. In addition, the liquid and the airflow are also sprayed oppositely in the nozzle with the tiny size, and the nozzle with the tiny size is very easy to be blocked when it is not working for a long time, affecting the quality effect of the product.

### SUMMARY

The present invention aims to provide an essential oil fragrance diffuser, which can achieve the advantage of preventing liquid from flowing out due to toppling. The ball rolling design is adopted and the mist hole is blocked based on the gravity principle, so that the liquid is prevented from flowing out. Meanwhile, the liquid on the channel is directly sprayed through the high-pressure airflow, the nozzle airflow is sprayed to the liquid from top to bottom, the nozzle is spaced from the liquid of the channel, and the liquid does not make contact with the micropores of the spray head, thereby preventing the viscous liquid from accumulating on the nozzle and blocking the nozzle when the product does not work for a long time, and affecting the quality and service life of the product, so as to solve the problem raised in the described background.

To achieve the above objectives, the present invention provides a technical solution as follows: an essential oil fragrance diffuser, including a shell, where
an inner cavity of the shell is provided with a support mechanism used for bearing a component, a power output air pressure mechanism is arranged on an outer side of the support mechanism, a liquid spraying mechanism is arranged inside the support mechanism, a silencing mechanism used for noise reduction processing is arranged at the top of the spraying mechanism, and a liquid storage mechanism is arranged at a bottom end of the silencing mechanism;
a fixed bracket in the support mechanism is connected inside the shell; and
the silencing mechanism includes a spray head main body pipe, the spray head main body pipe is mounted inside the fixed bracket, a first silencing member and a second silencing member are fixedly mounted on an inner side wall of the spray head main body pipe, respectively, and the first silencing member is arranged above the second silencing member.

Exemplarily, the liquid storage mechanism includes an essential oil bottle sealing ring, where the essential oil bottle sealing ring is placed at the bottom of an inner cavity of the spray head main body pipe, an inner cavity at the bottom of the spray head main body pipe is in threaded connection with an essential oil bottle, and the essential oil bottle sealing ring is located at a bottom end of the spray head main body pipe.

Exemplarily, the liquid spraying mechanism includes a spray head body, where the spray head body is mounted at the top of an inner cavity of the essential oil bottle, a spray core is mounted at the top of an inner cavity of the spray head body, the first silencing member and the spray head body are both internally provided with a ball body, and a baffle is fixedly mounted at the bottom of an outer side of the spray head body.

Exemplarily, a liquid inlet end of the spray head body is in communication with a liquid inlet pipe, and a bottom end of the liquid inlet pipe is provided with an inclined surface.

Exemplarily, the support mechanism further includes a switch and a circuit board, where the circuit board is fixedly mounted at the top of an outer side of the fixed bracket, and the switch is mounted on one side of the top of the circuit board.

Exemplarily, the air pressure mechanism includes an air pump, the air pump is connected to the outer side of the fixed bracket, and a battery is further mounted at the bottom of the outer side of the fixed bracket.

Exemplarily, an operating mechanism used for controlling spraying is arranged at the top of the fixed bracket, and an auxiliary mechanism used for sealing the spraying is arranged at the bottom of the outer side of the fixed bracket.

Exemplarily, the operating mechanism includes a key surface cover, where the key surface cover is mounted on an inner side wall of the top of the shell, and one end of the top of the key surface cover is connected to a spray head cover in a penetrating manner.

Exemplarily, the auxiliary mechanism includes a battery bottom cover, where the battery bottom cover is connected to a bottom end of the fixed bracket, and a spray head air inlet sealing rubber plug is mounted on one side of the spray head main body pipe.

Exemplarily, a bottom end of the spray head cover is in communication with a top end of the spray head main body pipe, and a hole is formed in the top of the spray head cover.

Compared with the prior art, the present invention has the beneficial effects:

The present invention has the advantages of preventing liquid from flowing out and reducing noise through a matching arrangement of the spray head main body pipe, the first silencing member, the spray head cover, the second silencing member, the spray head body, the spray core, and the ball. According to an anti-backflow protection structure, when a product topples over, a steel ball rolls to a mist outlet hole by utilizing a principle of gravity, the mist outlet hole is blocked, so that the liquid can be prevented from flowing out from the mist outlet hole. Meanwhile, the liquid on the channel is directly sprayed through the high-pressure airflow, the nozzle airflow is sprayed to the liquid from top to bottom, the nozzle is spaced from the liquid of the channel, and the liquid does not make contact with the micropores of the spray head, thereby preventing the viscous liquid from accumulating on the nozzle and blocking the nozzle when the product does not work for a long time, and affecting the quality and service life of the product. According to the mute design in the spray head, backflow and reflection of airflow are utilized, and a reverberation sound generated by the high-pressure sprayed gas fluid is weakened. In addition, after the kinetic energy of the airflow is weakened, the speed at which the mist is blown out is weakened, and the mist is gradually accumulated in the flowing process, so that the dense mist can be blown out with a slow effect. A spraying opening of the spray head body is provided with a blocking piece, and when the liquid of the bottle is reduced by a certain capacity, the high-pressure gas fluid can be prevented from being sprayed into the bottle to generate a hollow sound.

Additional features and advantages of the present invention will be set forth in the description which follows, and in part will be obvious from the description, or may be learned by implementing the present invention. The purpose and other advantages of the present invention may be implemented and obtained by a structure pointed out in the specification and drawings.

### BRIEF DESCRIPTION OF THE DRAWINGS

FIG. 1 is a schematic structural diagram of the present invention;

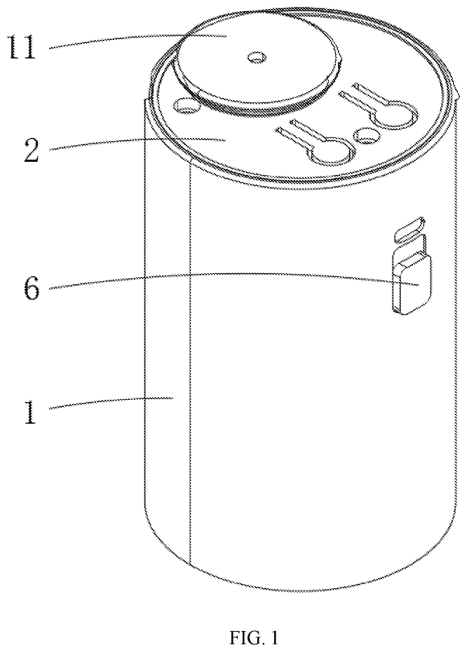

FIG. 2 is a schematic structural diagram of a battery bottom cover according to the present invention;

FIG. 3 is a schematic diagram of an internal structure of a shell according to the present invention;

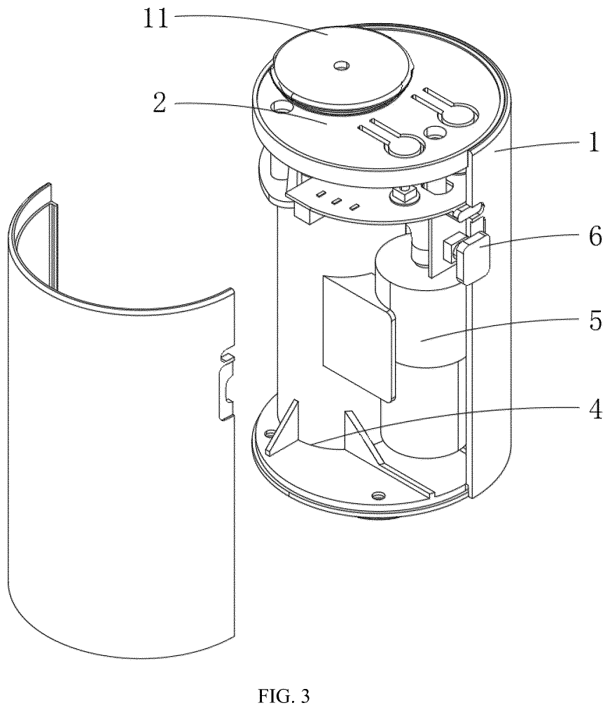

FIG. 4 is a schematic structural diagram of an air pump according to the present invention;

FIG. 5 is a schematic structural diagram of a fixed bracket according to the present invention;

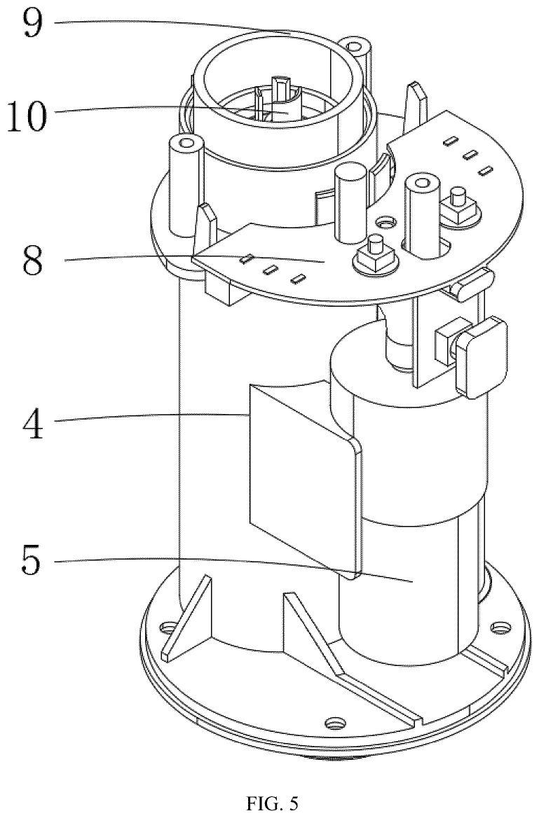

FIG. 6 is a schematic structural diagram of a battery according to the present invention;

FIG. 7 is a schematic structural diagram of an essential oil bottle according to the present invention;

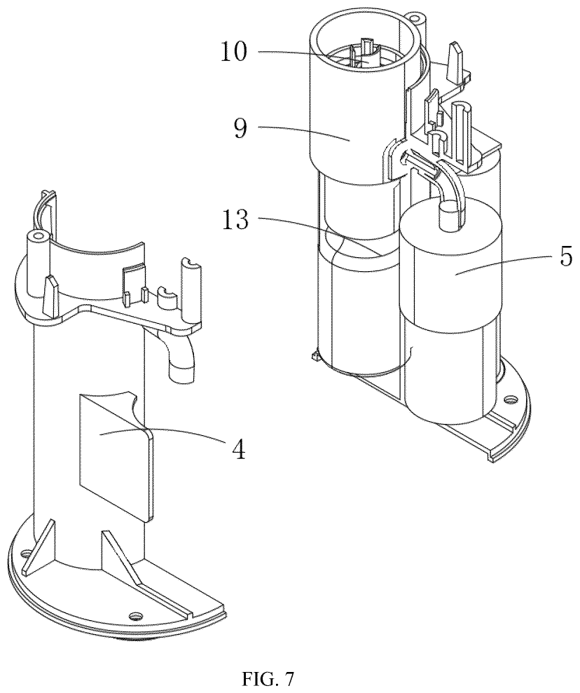

FIG. 8 is a schematic structural diagram of a first silencing member according to the present invention; and

FIG. 9 is a schematic structural diagram of a spray core according to the present invention.

In the figures: 1. shell; 2. key surface cover; 3. battery; 4. fixed bracket; 5. air pump; 6. switch; 7. battery bottom cover; 8. circuit board; 9. spray head main body pipe; 10. first silencing member; 11. spray head cover; 12. second silencing member; 13. essential oil bottle; 14. essential oil bottle sealing ring; 15. spray head body; 16. spray core; 17. spray head air inlet sealing rubber plug; 18. baffle.

### DETAILED DESCRIPTION OF THE EMBODIMENTS

The following clearly and completely describes the technical solutions in the embodiments of the present invention with reference to the accompanying drawings in the embodiments of the present invention. Apparently, the described embodiments are merely some rather than all of the embodiments of the present invention. All other embodiments obtained by those of ordinary skill in the art based on the embodiments of the present invention without creative efforts shall fall within the protection scope of the present invention.

The present invention provides an essential oil fragrance diffuser, including a shell 1 of the fragrance diffuser, where an inner cavity of the shell 1 is provided with a support mechanism used for bearing a component, a power output air pressure mechanism is arranged on an outer side of the support mechanism, a liquid spraying mechanism is arranged inside the support mechanism, a silencing mechanism used for noise reduction processing is arranged at the top of the spraying mechanism, and a liquid storage mechanism is arranged at a bottom end of the silencing mechanism; and a fixed bracket 4 in the support mechanism is connected inside the shell 1.

Specifically, the silencing mechanism includes a spray head main body pipe 9, the spray head main body pipe 9 is mounted inside the fixed bracket 4, a first silencing member 10 and a second silencing member 12 are fixedly mounted on an inner side wall of the spray head main body pipe 9, respectively, and the first silencing member 10 is arranged above the second silencing member 12.

Compared with the prior art, the mist volume effect generated by the structure in this embodiment is denser and finer than that of a product on the market, the sound is smaller, the mounting is easier, and the nozzle is not prone to being blocked.

Preferably,

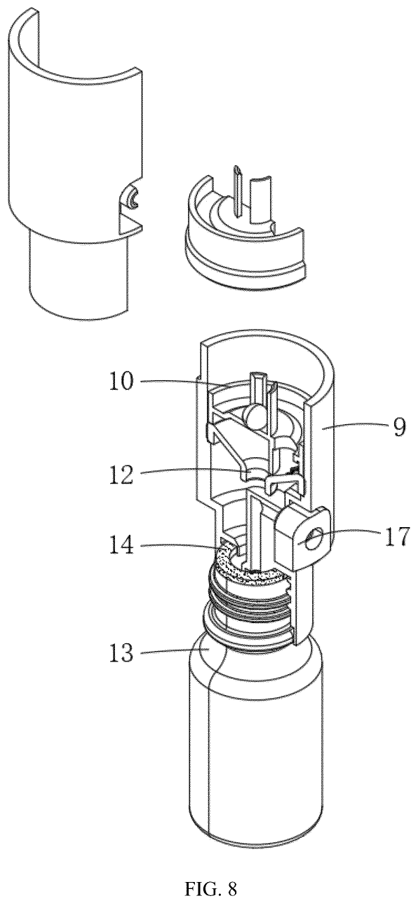

as shown in FIG. 8, the liquid storage mechanism includes an essential oil bottle sealing ring 14, where the essential oil bottle sealing ring 14 is placed at the bottom of an inner cavity of the spray head main body pipe 9, an inner cavity at the bottom of the spray head main body pipe 9 is in threaded connection with an essential oil bottle 13, and the essential oil bottle sealing ring 14 is located at a bottom end of the spray head main body pipe 9, so that the sealing performance of connection is improved through the essential oil bottle sealing ring 14 when the essential oil bottle 13 is mounted, and the connection manner adopts a threaded rotary connection manner to enable the upper and lower parts to be in communication and fixed.

Further,

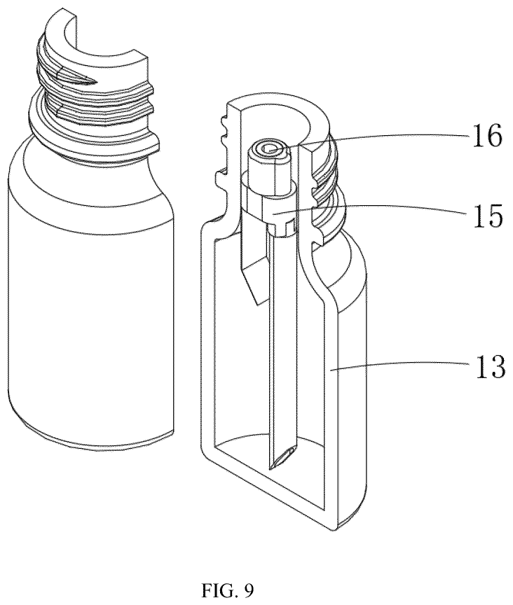

as shown in FIG. 8 and FIG. 9, the liquid spraying mechanism includes a spray head body 15, where the spray head body 15 is mounted at the top of an inner cavity of the essential oil bottle 13, a spray core 16 is mounted at the top of an inner cavity of the spray head body 15, the first silencing member 10 and the spray head body 15 are both internally provided with a ball body, and a baffle 18 is fixedly mounted at the bottom of an outer side of the spray head body 15. The spray head body 15 is matched with the spray core 16, so that the liquid on the channel can be directly sprayed through the high-pressure airflow, the nozzle airflow is sprayed to the liquid from top to bottom, the nozzle is spaced from the liquid of the channel, and the liquid does not make contact with the micropores of the spray head, thereby preventing the viscous liquid from accumulating on the nozzle and blocking the nozzle when the product does not work for a long time, and affecting the quality and service life of the product.

Based on the above, since a nozzle spraying direction of the spray head body 15 is downward, and the nozzle is spaced from the liquid of the channel, the liquid is effectively prevented from entering the nozzle and passing through an air channel to reach an air pump, and breaking air pump parts; and

secondly, a spraying opening of the spray head body 15 is provided with a baffle 18, and when the liquid of the bottle is reduced by a certain capacity, the high-pressure gas fluid can be prevented from being sprayed into the bottle to generate a hollow sound.

According to the structure, the low-air-amount air pressure mechanism is adopted and matched with the spray core 16, so that the fragrance diffuser can be used for a long time, and the noise generated by the air pressure mechanism is lower. The spray core 16 of the spray head acts to enable the airflow to pass through the tiny hole diameter (pressurizing hole) of the spray core 16, so that the airflow is converted into a high-speed air column and is sprayed to the liquid in the pipeline, the liquid is instantly converted into tiny particles, and the hole diameter of the particle is 0.1-0.3 mm. However, on the market, the pressurizing hole and the oil outlet hole are sprayed oppositely, and in this design, only the pressurizing hole is needed.

In the drawings:

as shown in FIG. 9, a liquid inlet end of the spray head body 15 is in communication with a liquid inlet pipe, and a bottom end of the liquid inlet pipe is provided with an inclined surface, so as to facilitate the flow of the liquid, and the liquid is sprayed out in a drainage manner in a negative pressure state.

### Still Further,

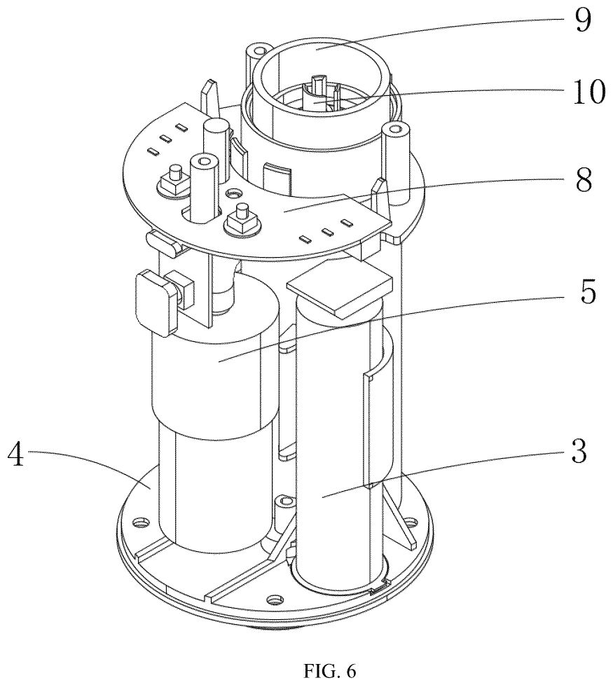

as shown in FIG. 6, the support mechanism further includes a switch 6 and a circuit board 8, where the circuit board 8 is fixedly mounted at the top of an outer side of the fixed bracket 4, and the switch 6 is mounted on one side of the top of the circuit board 8 and is used for opening and closing and controlling the operation of the fragrance diffuser.

In the drawings:

as shown in FIG. 6, the air pressure mechanism includes an air pump 5, the air pump 5 is connected to the outer side of the fixed bracket 4, and a battery 3 is further mounted at the bottom of the outer side of the fixed bracket 4, so that the liquid can be atomized and sprayed out under the impact of air pressure through negative pressure.

In addition,

an operating mechanism used for controlling spraying is arranged at the top of the fixed bracket 4, and an auxiliary mechanism used for sealing the spraying is arranged at the bottom of the outer side of the fixed bracket 4.

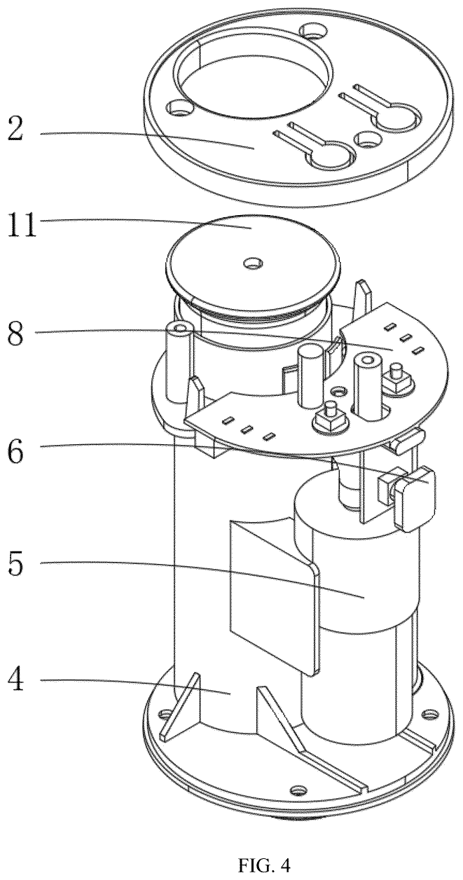

As shown in FIG. 4, the operating mechanism includes a key surface cover 2, the key surface cover 2 is mounted on an inner side wall of the top of the shell 1, and one end of the top of the key surface cover 2 is connected to a spray head cover 11 in a penetrating manner. The key surface cover 2 can be used for regulating and controlling the operation of the fragrance diffuser, and the spray head cover 11 is beneficial to protecting a spraying position, so that sprayed atomized matters are sprayed out according to a preset position.

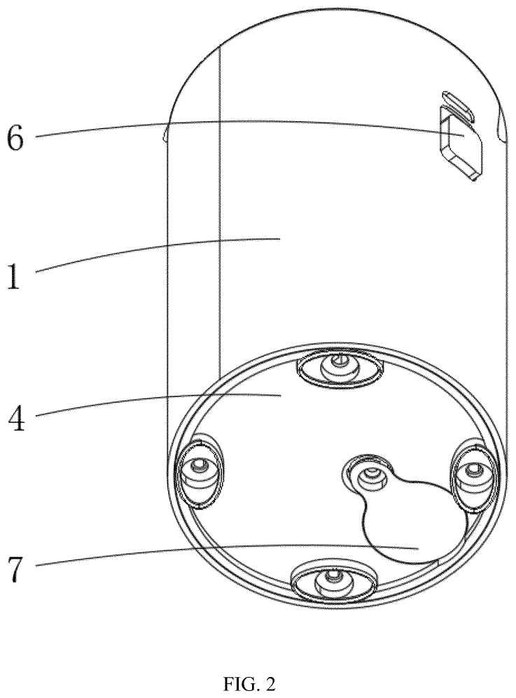

As shown in FIG. 2 and FIG. 8, the auxiliary mechanism includes a battery bottom cover 7, where the battery bottom cover 7 is connected to a bottom end of the fixed bracket 4, and a spray head air inlet sealing rubber plug 17 is mounted on one side of the spray head main body pipe 9 and is used for protecting the battery 3, and meanwhile, disassembly and assembly and replacement are facilitated.

As shown in FIG. 4, a bottom end of the spray head cover 11 is in communication with a top end of the spray head main body pipe 9, and a hole is formed in the top of the spray head cover 11, so that atomized gas can be sprayed out from the interior of the spray head cover through the hole.

As shown in the figures, the battery 3 is used for supplying power to an electrical element and an electronic element in the embodiment, and the structure convenient to disassemble and assemble is adopted to facilitate quick replacement when the subsequent electric quantity is exhausted, thereby ensuring normal use. According to the air pump 5, the pipeline is in communication with the spray head, the high-speed flowing gas forms negative pressure at a liquid inlet, the essential oil is rapidly sucked into the liquid inlet under the action of the pressure difference, and it can be known in combination with the drawings. Then, the essential oil is dispersed by the high-speed flowing gas to form mist-shaped oil drops of the small particles, the mist-shaped oil drops are discharged along with the gas from a mist outlet position, and are atomized by passing through the spray head body 15 and the internal flow channel, and meanwhile, the gas is pressurized when the mist-shaped oil drops penetrate through the spray core 16, the flow velocity of the gas is increased, and the gas is continuously upward;

according to the above, in the continuous working process, the mist-shaped oil drops can be driven by gas to go upwards until passing through the second silencing member 12 and the first silencing member 10 and are discharged through the spray head cover 11 and the formed hole, and since the mist-shaped oil drops pass through the second silencing member 12 and the first silencing member 10, generated noise can collide and counteract between the second silencing member 12 and the first silencing member 10, and therefore the noise reduction effect is achieved; and

in order to prevent liquid from flowing out due to toppling, two groups of ball bodies are used, and during toppling, the ball bodies roll to the mist outlet hole based on their own gravity, so that the mist outlet hole is blocked, and the liquid can be prevented from flowing out of the mist outlet hole, so that the situation that the liquid flows out due to toppling is prevented.

Although the embodiments of the present invention have been shown and described, it will be appreciated by those of ordinary skill in the art that various changes, modifications, substitutions, and variations can be made to these embodiments without departing from the principle and spirit of the present invention, and the scope of the present invention is defined by the appended claims and their equivalents.

What is claimed is:

1. An essential oil fragrance diffuser, comprising a shell (1) of the fragrance diffuser, wherein an inner cavity of the shell (1) is provided with a support mechanism used for bearing a component, the support mechanism comprises a fixed bracket (4), and the fixed bracket (4) is connected inside the shell (1);
a power output air pressure mechanism is arranged on an outer side of the support mechanism, a liquid spraying mechanism is arranged inside the support mechanism, a silencing mechanism used for noise reduction processing is arranged at the top of the spraying mechanism, and a liquid storage mechanism is arranged at a bottom end of the silencing mechanism; and
the silencing mechanism comprises a spray head main body pipe (9), the spray head main body pipe (9) is mounted inside the fixed bracket (4), a first silencing member (10) and a second silencing member (12) are fixedly mounted on an inner side wall of the spray head main body pipe (9), respectively, and the first silencing member (10) is arranged above the second silencing member (12).

2. The essential oil fragrance diffuser according to claim 1, wherein the liquid storage mechanism comprises:
an essential oil bottle sealing ring (14);
the essential oil bottle sealing ring (14) is placed at the bottom of an inner cavity of the spray head main body pipe (9), and the essential oil bottle sealing ring (14) is located at a bottom end of the spray head main body pipe (9); and
an inner cavity at the bottom of the spray head main body pipe (9) is in threaded connection with an essential oil bottle (13).

3. The essential oil fragrance diffuser according to claim 2, wherein the liquid spraying mechanism comprises:
a spray head body (15); and
the spray head body (15) is mounted at the top of an inner cavity of the essential oil bottle (13), a spray core (16) is mounted at the top of an inner cavity of the spray head body (15), the first silencing member (10) and the spray head body (15) are both internally provided with a ball body, and a baffle (18) is fixedly mounted at the bottom of an outer side of the spray head body (15).

4. The essential oil fragrance diffuser according to claim 3, wherein a hole diameter of the spray core (16) is 0.1-0.3 mm.

5. The essential oil fragrance diffuser according to claim 3, wherein a liquid inlet end of the spray head body (15) is in communication with a liquid inlet pipe, and a bottom end of the liquid inlet pipe is provided with an inclined surface.

6. The essential oil fragrance diffuser according to claim 1, wherein the support mechanism further comprises a switch (6) and a circuit board (8), the circuit board (8) is fixedly mounted at the top of an outer side of the fixed bracket and the switch (6) is mounted on one side of the top of the circuit board (8).

7. The essential oil fragrance diffuser according to claim 1, wherein the air pressure mechanism comprises an air pump (5), the air pump (5) is connected to the outer side of the fixed bracket (4), and a battery (3) is further mounted at the bottom of the outer side of the fixed bracket

8. The essential oil fragrance diffuser according to claim 1, wherein an operating mechanism used for controlling spraying is arranged at the top of the fixed bracket (4), and an auxiliary mechanism used for sealing the spraying is arranged at the bottom of the outer side of the fixed bracket (4).

9. The essential oil fragrance diffuser according to claim 8, wherein the operating mechanism comprises a key surface cover (2), the key surface cover (2) is mounted on an inner side wall of the top of the shell (1), and one end of the top of the key surface cover (2) is connected to a spray head cover (11) in a penetrating manner.

10. The essential oil fragrance diffuser according to claim 8, wherein the auxiliary mechanism comprises a battery bottom cover (7), the battery bottom cover (7) is connected to a bottom end of the fixed bracket (4), and a spray head air inlet sealing rubber plug (17) is mounted on one side of the spray head main body pipe (9).

11. The essential oil fragrance diffuser according to claim 9, wherein a bottom end of the spray head cover (11) is in communication with a top end of the spray head main body pipe (9), and a hole is formed in the top of the spray head cover (11).
